(19) United States
(12) Patent Application Publication
(10) Pub. No.: US 2025/0029718 A1
(43) Pub. Date: Jan. 23, 2025

(54) LOCATING THE DISTAL END OF A MEDICAL INSTRUMENT

(71) Applicant: Siemens Healthineers AG, Forchheim (DE)

(72) Inventors: Alois Regensburger, Poxdorf (DE); Marcus Pfister, Bubenreuth (DE)

(21) Appl. No.: 18/771,346

(22) Filed: Jul. 12, 2024

(30) Foreign Application Priority Data
Jul. 19, 2023 (DE) 10 2023 206 855.9

(51) Int. Cl. G16H 40/63; A61B 34/20; G06T 7/00

(52) U.S. Cl. CPC G16H 40/63; G06T 7/0002; A61B 2034/2055; G06T 2207/10081; G06T 2207/10088

(57) ABSTRACT

Systems and methods for more reliability located the distal end of a medical instrument. A data set is provided that describes a course of a main duct, of a first branch from the main duct, and of a second branch from the main duct. A course of the medical instrument in the main duct and in one of the first or second branches is moreover detected. The course of the main duct and of the two branches is brought into register with the course of the medical instrument. A first similarity measure relating to the end portion and the first branch and a second similarity measure of the end portion and the second branch are then calculated. An item of location information about the distal end of the medical instrument is determined from the similarity measures.

### CROSS REFERENCE TO RELATED APPLICATIONS

This application claims the benefit DE 10 2023 206 855.9 filed on Jul. 19, 2023, which is hereby incorporated by reference in its entirety.

### FIELD

Embodiments relate to a method for locating a distal end of a medical instrument.

### BACKGROUND

It is necessary in some fields of medicine to investigate or treat vessels with instruments, to which end the particular instrument (e.g., catheter or guidewire) has to advance into a vessel lumen. Such cannulation is frequently carried out with observation by an imaging modality with the intention being to establish where the tip of the medical instrument is situated in the particular vessel.

Bile ducts may also be investigated by way of cannulation (ERCP cannulation: creation of an access to the bile ducts in the course of an endoscopic retrograde cholangio-pancreatography). This for example involves advancing a so-called guidewire into a bile duct.

It is difficult for the physician to decide during an ERCP cannulation whether the tip of the guidewire is situated precisely in the common bile duct or in the pancreatic duct (or “ductus pancreaticus”). Under certain circumstances, the tip of the guidewire may however also be unintentionally advanced. The injection of contrast agent must for example be avoided during this step of the procedure as this is associated with an elevated risk of complications (for example pancreatitis). In an X-ray image it is thus only the guidewire, but not the bile duct anatomy that is visible. This problem is of particular clinical relevance since inadvertent cannulation of the pancreatic duct or injection of contrast agent into the latter significantly increases the risk of complications.

Given that some 2,000,000 ERCP procedures are carried out annually and there is a complication rate of 5 to 10% of the patients, with a considerable proportion of the complications occurring during cannulation, improved guidance of cannulation may potentially avoid a very large and costly number of complications. Virtually all ERCP procedures are carried out with X-ray imaging but other imaging modalities are in principle usable too.

A conventional procedure during cannulation is for the physician to advance the guidewire further as soon as no resistance is any longer felt. Once the guidewire has been advanced a few centimeters further, experienced physicians may then see from the direction of the guidewire whether the duct is probably the pancreatic duct or the common bile duct. Inexperienced physicians and, in the case of unexpected patient anatomy, even experienced physicians, inject contrast agent in order to be able to see the anatomy of the bile duct around the guidewire. Unintended cannulation of the pancreatic duct leads to a distinct increase in the risk of complications that means that under certain circumstances preventive treatment for pancreatitis, such as placing a stent in the pancreatic duct, has to be performed at the same time.

### BRIEF SUMMARY AND DESCRIPTION

The scope of the embodiments is defined solely by the appended claims and is not affected to any degree by the statements within this summary. The present embodiments may obviate one or more of the drawbacks or limitations in the related art.

Embodiments locate the distal end of a medical instrument more reliably.

Embodiments accordingly provides a method for locating a distal end of a medical instrument by providing a data set that describes a course of a main duct, of a first branch from the main duct, and of a second branch from the main duct (from a common branching point), detecting a course (e.g., line) of the medical instrument in the main duct and in one of the first or second branches, bringing the course of the main duct and of the two branches into register with the course of the medical instrument, calculating a first similarity measure of a portion of the course of the medical instrument in the first or second branch with a first portion of the course of the first branch and a second similarity measure of the portion of the course of the medical instrument in the first or second branch with a second portion of the course of the second branch, and determining an item of location information about the portion (e.g., distal end region) of the course of the medical instrument from the two similarity measures.

A method for locating a distal end of a medical instrument is accordingly provided. The medical instrument may be, for example, a catheter, a guidewire, a sphincterotome, an endoscope (e.g., “SpyGlass”) or a part thereof. For example, it may thus be a guidewire of a sphincterotome. The distal end of this medical instrument is to be located. This means that an item of location information about the distal end or about a portion of the medical instrument at the distal end is to be obtained.

To this end, a data set is first provided that describes a course of a main duct, of a first branch from the main duct and of a sidebranch from the main duct. Such a constellation occurs, for example, in the case of vessel branching. The main duct for example divides into two secondary ducts, i.e., branches. This is for example the case for the bile duct channel that leads from the duodenum into the pancreas and conducts the bile. Viewed in the direction of flow, the pancreatic duct and the common bile duct open into a common channel and the latter then opens into the duodenum.

The method may, however, also be used for other bifurcations, if for example a main duct has a plurality of successive branches and itself continues onward. The branches then do not branch at one point, as in the first example, but instead each branch off from the main duct at successive points.

A data set is provided that describes the respective geometry (main duct with two branches). In the simplest case, this geometry may be described with three lines arranged in a Y-shape. The data set should represent the actual geometry of the main duct and the branches as accurately as possible. For example, angles and lengths of the respective components should be stated.

In a further step of the method, the course of the medical instrument in the main duct and in one of the first or second branches is detected. This means that the medical instrument is situated both in the main duct and in one of the branches. For example, the distal end of the medical instrument is situated in one of the branches. The medical instrument (e.g., guidewire) has for example a linear course. In this case, the line of this course may be detected, for example, by an appropriate imaging modality.

In a further step, the course of the main duct and of the two branches is brought into register with the course of the medical instrument. This means that the detected actual course of the medical instrument is for example brought into register with the course of a vessel bifurcation (with main duct and two branches) that has previously been determined and provided in the data set. For example, the images of the two courses are slid over one another as congruently as possible.

In a further step, the first similarity measure of a portion of the course of the medical instrument in the first or second branch with a first portion of the course of the first branch is calculated. This means that it is investigated how similar the course of the medical instrument at the distal end is to the course of the first portion of the data set. A second similarity measure of the portion of the course of the medical instrument in the first or second branch with the second portion of the course of the second branch may be determined analogously. This also means that it is determined how similar the course of the distal end of the medical instrument is to the course of the second branch. Depending on whether the medical instrument is situated in the first or second branch, the similarity measures are correspondingly higher or lower. If, for example, the distal end of the medical instrument is situated at the second branch, the course of the medical instrument is more similar to the course of the second branch than to the course of the first branch. This greater similarity is reflected in the similarity measures.

Finally, the method involves determining an item of location information about the portion of the course of the medical instrument from the two similarity measures. This portion of the course of the medical instrument represents the distal end or the distal end region of the medical instrument. The location information may for example be determined such that the distal end, i.e., the relevant portion of the course of the medical instrument, is assigned to one of the two branches on the basis of the two similarity measures. If the course of the medical instrument at its distal end (in the present document synonymous with “distal end region”) is somewhat more similar to the course of the first branch, the distal end is assigned to the first branch. This assignment gives rise to the location information that the distal end is situated in the first branch. The location information need not, however, be clear-cut. Instead, the location information may also be a probability with which the distal end of the medical instrument is situated in one of the branches.

Embodiments make it possible to obtain an item of location information about the distal end of a medical instrument without for example contrast agent administration being necessary.

One embodiment provides that the data set is created from at least one X-ray image, MRI (magnetic resonance tomography) image, a computed tomography (CT) image, and/or ultrasonogram. The geometry of the main duct and of the two branches may thus be obtained in advance by appropriate imaging. In medical applications, vessel branching may, for example, be reliably identified using the stated techniques.

A further embodiment provides that a length of the medical instrument is detected and the method is only carried out once the length of the medical instrument is greater than a length of the course of the main duct of the data set. The medical instrument is typically firstly inserted into a main duct, from where penetration into one of the branches is attempted. It only makes sense to make a statement as to the branch into which the medical instrument has penetrated once the latter has passed completely through the main duct. This means that the length of the medical instrument must likewise be greater than the length of the main duct. For example, a guidewire is guided from a duodenoscope into the common channel and then onward into the pancreatic duct. On deployment of the guidewire from the duodenoscope, the deployed length of the guidewire may be detected by appropriate marks on the guidewire. Once a certain length has been reached, it may be assumed that the common channel has been completely passed through and the distal end of the guidewire is situated in one of the branches. Only then does it make sense to carry out the method presented here.

In a further embodiment, the two courses are respective 2D projections. This means that each of the courses is represented as a two-dimensional shape. The two-dimensional representation reduces computing and memory requirements. However, since they are projections, account should be taken of any projection angles that may result in distortion.

A further embodiment provides that the course of the main duct and of the two branches is saved in the data set as concatenated line portions. Here too, it is not the complete geometry of the main duct and of the two branches that is stored but instead just corresponding line portions, whereby computing and memory requirements are again reduced. A typical stored data set may thus for example represent a Y-shaped course structure, namely a line portion of the main duct and two further line portions that represent the two branches.

In another embodiment, the similarity measures are in each case based on an angle of the portion of the course of the medical instrument in the first or second branch in a predetermined coordinate system. The coordinate system may, for example, be oriented relative to a patient's spinal column. While in an AP (anterioposterior) X-ray projection, the pancreatic duct generally runs crosswise relative to the backbone, the common bile duct tends to run parallel to the backbone. Depending on the duct into which the medical instrument has penetrated, a different angle is thus obtained. This angle may be used as a similarity measure. The angle or orientation of the distal end of the medical instrument thus itself often already provides an indication of the location of the distal end, for example the bifurcation that is occupied.

Alternatively, or additionally, the similarity measures may in each case be based on a Hausdorff metric between the portion of the course of the medical instrument in the first or second branch and the respective portion of the course of the respective branch. A portion of the distal end region of the medical instrument relative to the respective branch is as it were calculated. For the calculation, the two courses are the respective subsets to which the Hausdorff metric is applied. The smaller the distance of the medical instrument from the respective course of the branch, the more similar are the two courses or the more probably is the medical instrument situated in the respective branch.

An extended embodiment provides that, on the basis of the similarity measures, the location information is calculated as a probability with which the medical instrument is situated in the first or second branch, and this probability or a value based thereon is made available to a user. A similarity measure may thus be converted into a probability that a user may more readily interpret. The user is then for example informed that the distal end of the medical instrument is situated with a probability of 80% in the pancreatic duct and with a probability of only 20% in the common bile duct. The user may thus better estimate their subsequent course of action.

One embodiment may furthermore provide that a risk measure is determined as a function of a length of the medical instrument in the first or second branch and the similarity measures and is made available to a user. A risk may for example increase if the medical instrument is inserted further into the respective branch. For example, the risk of inflammation may increase. It is thus favorable for the user to be provided with a corresponding risk factor while guiding the medical instrument.

In a further embodiment, detection of the course of the medical instrument is carried out automatically by an imaging modality and a data processing facility. For example, the imaging modality obtains corresponding X-ray, MRI, CT images, or ultrasonograms that are further processed by the data processing facility. The data processing facility may for instance extract or detect the course of the images as a line. The data processing facility may furthermore also be configured for image recognition in order to recognize predetermined line courses.

As already indicated above, in a further embodiment, a length of the medical instrument may be detected independently of the detection of the course of the medical instrument and used for calculating the similarity measures. For example, the length of the medical instrument may be determined with the assistance of marks thereon. The actual length of the medical instrument may provide information about distortion that may occur during image acquisition of the medical instrument. Such distortion may have to be taken into account for calculating the similarity measure.

Another development of the method according to embodiments may provide that a respective deformation of the first and/or second branch caused by the medical instrument is determined using a known deformation method, such as for example EVAR guidance, and, with the assistance of the deformation(s), a probability with which the medical instrument is situated in the first or second branch is calculated (EVAR=Endovascular Aortic Repair; approaches are known in EVAR guidance that involve deforming a superimposed vessel on the basis of a shape, visible in the imaging, of a catheter or medical instrument situated in the vessel). Using this deformation method, it is for example possible to determine deformation of a vessel when a medical instrument penetrates therein. This deformation may then also be used to determine the location information about the medical instrument or the distal end thereof.

The above object is also achieved according to embodiments by a system including a medical instrument, a memory facility configured to provide a data set that describes a course of a main duct, of a first branch from the main duct, and of a second branch from the main duct, an imaging modality configured to detect a course of the medical instrument in the main duct and in one of the first or second branches, a data processing facility configured for bringing the course of the main duct and of the two branches into register with the course of the medical instrument, calculating a first similarity measure of a portion of the course of the medical instrument in the first or second branch with a first portion of the course of the first branch and a second similarity measure of the portion of the course of the medical instrument in the first or second branch with a second portion of the course of the second branch, and determining an item of location information about the portion of the course of the medical instrument from the two similarity measures.

The advantages and possible variants recited above in connection with the described method also apply mutatis mutandis to the system according to embodiments. The individual method steps may accordingly also be interpreted as functional features of corresponding system.

The memory facility may be a data memory that may store and, when required, return the corresponding data records, for example about line courses of the main duct and the branches. Under certain circumstances, the memory facility has its own processor that controls storage processes.

The medical instrument may be, for example, part of a catheter, a sphincterotome or a guidewire. The medical instrument may also be the entire catheter, the entire sphincterotome or the entire guidewire.

In one embodiment, the imaging modality includes an X-ray apparatus, an MRI apparatus, or an ultrasonography apparatus. The X-ray apparatus may for example be a C-arm device or an angiography device.

Embodiments furthermore also provides a computer program or computer program product including commands that, on execution by the above-stated system, cause the latter to carry out a method likewise recited above.

For application cases or application situations that may arise during the method and are not described explicitly here, provision may be made for an error message and/or a request to submit user feedback to be output and/or a default setting and/or a predetermined initial state to be set.

Independent of the grammatical term usage, individuals with male, female or other gender identities are included within the term.

### BRIEF DESCRIPTION OF THE FIGURES

FIG. 1 depicts a schematic view of a system according to an embodiment.

FIG. 2 depicts is a schematic view of a course of a main duct and two branches and of a course of a medical instrument according to an embodiment.

FIG. 3 depicts the two courses of FIG. 2 brought into register with one another according to an embodiment.

FIG. 4 depicts a block diagram of an embodiment of a method.

### DETAILED DESCRIPTION

Embodiments are based on the concept that, once a data set, for example containing the bile ducts, has been registered, an automatic evaluation is carried out as to whether the tip of the guidewire visible in the X-ray image is probably situated in the pancreatic duct or in the common bile duct.

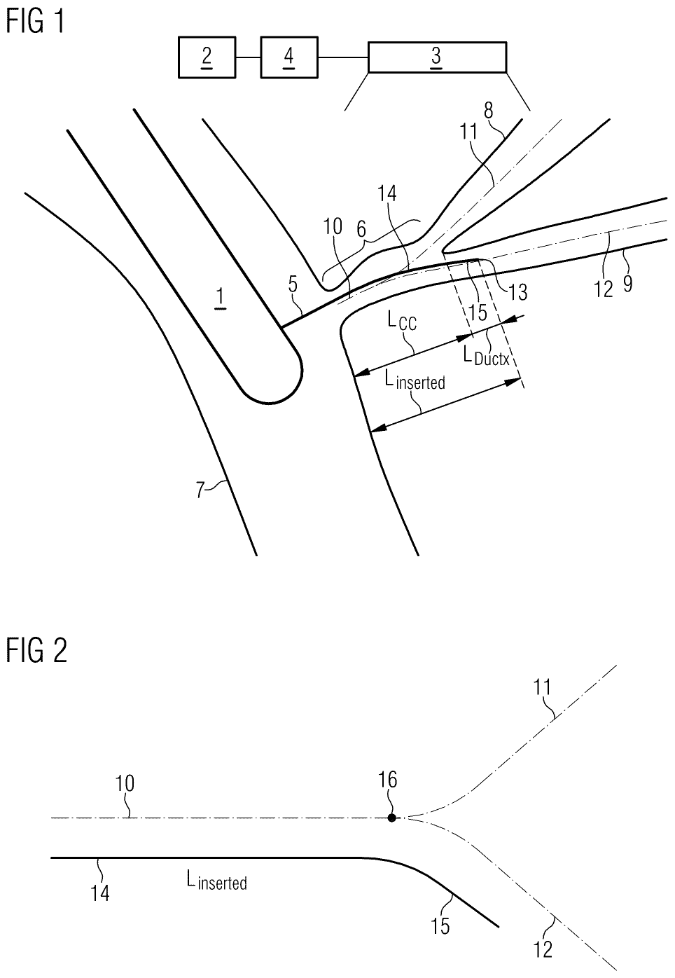

The embodiment of FIG. 1 schematically depicts a system according to embodiments including a medical instrument 1, a memory facility 2, an imaging modality 3, and a data processing facility 4. The medical instrument 1 may be, for example, a duodenoscope from which a guidewire 5 is deployable.

The medical instrument 1 is situated in the duodenum, specifically at the inlet of the gall bladder and pancreas, where a common channel 6 opens into the duodenum 7. On the opposite side from the mouth, the common channel 6 divides into the common bile duct 8 and the pancreatic duct 9. In general terms, the common channel 6 may be denoted the main duct, the common bile duct 8 the first branch, and the pancreatic duct 9 the second branch.

The course of the main duct 6 and of the two branches 8 and 9 may be represented in stylized manner by lines (e.g., midlines). The corresponding line portions may be denoted as follows: main duct midline 10, first branch midline 11 (e.g., of the common bile duct) and second branch midline 12 (e.g., of the pancreatic duct). The three line portions 10, 11, and 12 overall form a Y-shaped structure and characterize a bifurcation.

In the example of FIG. 1, the guidewire 5 is inserted through the common channel (main duct 6) into the pancreatic duct (second branch 9). The entire inserted length L_{inserted} extends from the mouth of the main duct 6 up to the tip or the distal end 13 of the guidewire 5. Of this entire inserted length L_{inserted}, one part 14 of length L_{CC} is situated in the common channel (main duct 6). The end portion 15 of the guidewire 5 has the length L_{Ductx}.

The challenge is now to generate an item of location information about which of the two branches 8 and 9 the end portion of the medical instrument 1 or guidewire 5 is situated in.

The imaging modality for detecting an actual course of the medical instrument 1 to 5 in the main duct 6 and in one of the first branch 8 and the second branch 9 may take the form of an X-ray device, MRI device, CT device, ultrasonography device or the like. The imaging modality 3 thus provides an image for example of the guidewire 5. Connected to the imaging modality 3 there is a data processing facility 4 that may process the one or more images from the imaging modality 3. For example, an image processing algorithm that recognizes the guidewire 5 and converts it into a line could be implemented on the data processing facility.

The system further includes the memory facility 2, with which at least one data set that describes a course of the main duct 6, of the first branch 8 and of the second branch 9 may be provided. The memory facility 2 supplies the data set to the data processing facility 4. The latter in turn brings the course of the main duct 6 and of the two branches 8, 9 into register with the course of the medical instrument 1 or 5. The course of the guidewire 5 is, for example, brought into register with one or more of the midlines 10, 11, and 12. The data processing facility 4 is furthermore capable of calculating a first similarity measure of the end portion 15 with a first portion of the course of the first branch 8. The data processing facility 4 may furthermore also calculate a second similarity measure that reflects the similarity of the end region or end portion 15 of the guidewire 5 with a second portion of the course of the second branch 9. Using these similarity measures, it is thus possible to obtain an objective quantity that indicates whether the end portion 15 or distal end 13 of the guidewire 5 is situated in the first branch 8 or the second branch 9. As a function of the similarity measures, the data processing facility 4 may accordingly also determine an item of location information that provides information about actual location of the end portion 15 of the guidewire 5.

One essential aspect is bringing a previously obtained data set that describes the course of the main duct 6 and of the two branches 8, 9 into register with the capture that shows the course of the medical instrument 1. The center- or midlines of the individual portions (main duct, first branch, second branch) may be segmented in the data set. A similarity measure is then obtained that for example reflects a distance of the end portion 15 from one of the midlines 11, 12, i.e., the distance of the guidewire 5 from one of the registered midlines of the branches. On the basis of this similarity measure, an estimate is then made as to which of the two branches the medical instrument 1 or guidewire 5 is located in (with what probability), that is equivalent to an item of location information.

A 2D X-ray system, for example a C-arm is substantially sufficient as the imaging modality.

In one specific embodiment, largely similarly to the example of FIG. 1, a guidewire or catheter (generally a medical instrument) has been inserted into a cavity (e.g., bile duct, vessel) of a patient. The hollow organ contains no contrast agent and is not visible to X-rays, at least not in the X-ray system used. The loaded image data set (e.g., MRCP) contains visible bile ducts represented in an at least stylized manner. The bile ducts are segmented (at least centerline segmentation) in a subregion of relevance to cannulation.

The current course and the length of the guidewire pushed into the bile duct system are determined. This is carried out, for example, by X-ray imaging. The length of the visible guidewire may be determined from the generally readily visible wall of the duodenum 7. From the wall of the duodenum 7 onward, the guidewire 5 is situated within the bile ducts that are not visible to X-rays. It is assumed as an approximation that the X-ray projection axis is perpendicular to the axis of that part of the guidewire 5 that is situated within the bile ducts. For the purposes of the application described here, this approximation is usually adequate.

The aim is to automatically obtain an item of location information as to which of the two bile duct bifurcations the front part (end portion 15) of the guidewire 5 is probably situated in. This location information may be provided automatically by an algorithmic evaluation. To this end, the line visible to X-rays of the end portion 15 is for example compared with the corresponding subportions of the center- or midlines of the two branches 8 and 9 (pancreatic duct and common bile duct).

The algorithmic evaluation for obtaining the location information may be carried out according to the following embodiment. The length of the inserted guidewire portion L_{inserted} (cf. FIG. 2) is firstly (automatically) determined. If this length L_{inserted} exceeds the length L_{CC} of the common channel 6, i.e., the guidewire 5 has also penetrated into one of the branches 8, 9, the method for automatically determining the location information makes sense and the following steps are carried out.

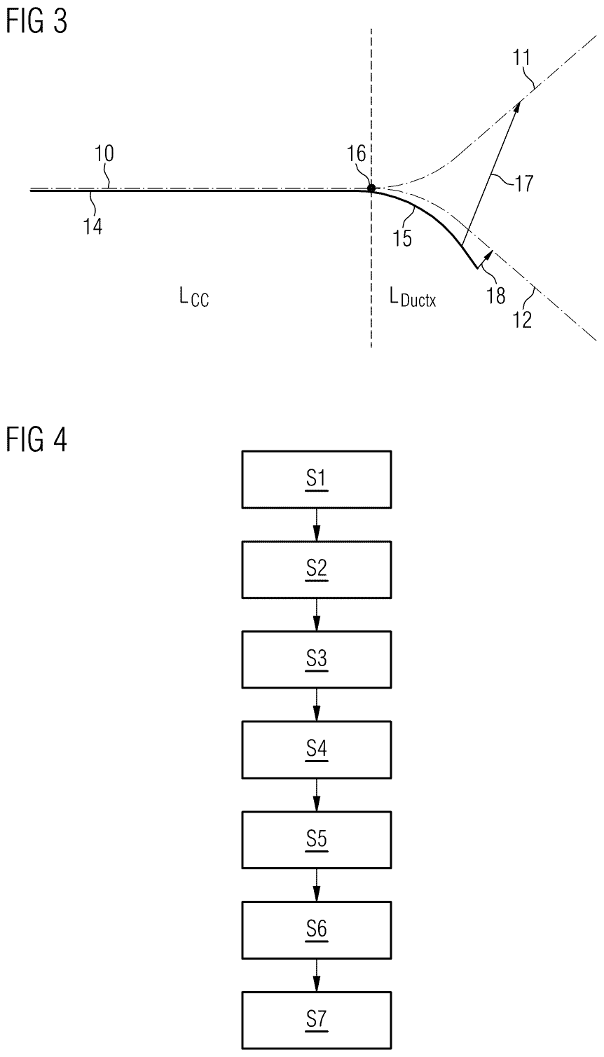

According to FIGS. 2 and 3, the entire guidewire line portion 14, 15 (length L_{inserted}) is brought into register with the main duct midline 10. This means that the course of the guidewire characterized by the line portions 14, 15 is slid onto the main duct midline 10. The projection of the inserted guidewire 5 is now divided into the main duct portion 14 (length L_{CC}) and the end portion 15 of the guidewire 5. To this end, a branch point is determined at which the main duct midline 10 divides into the first branch midline 11 and the second branch midline 12. The portion of the inserted guidewire up to the bifurcation point 16 that has been brought into register corresponds to the main duct portion 14 of the guidewire, and the portion from the bifurcation point 16 that has been brought into register onward corresponds to the end portion 15 of the guidewire (length_{Ductx}).

The course of the medical instrument in the first or second branch 8, 9, i.e., of the end portion 15, is then compared with the courses of the two branches 8, 9, i.e., of the line portions 11, 12. Similarity measures may to this end be determined using the following calculation approaches or combinations thereof: calculation or comparison of the angles/orientations of the respective line portions; calculation of line distances for example with the assistance of the generalized Hausdorff metric (Hausdorff distance).

The similarity measures may be used as the basis for determining a probability of probing of the one of the two branches 8, 9. A risk evaluation may or additionally also be determined from the determined length_{Ductx}. This is because the further the advance into the incorrect channel, the greater may be a corresponding risk of inflammation.

FIG. 1 may once again be considered for another embodiment in which the length_{CC} of the guidewire portion in the main duct (main duct portion 14) is determined. This may for example be approximately equated to the length of the main channel (“common channel”) in the segmentation of the preinterventional data set. In the event of major lateral expansion of the main duct or a major visible curvature of the guidewire in the X-ray image, the applicant's “EVAR guidance” methods may be used to determine length_{Ductx} and length_{CC}. The length of the guidewire portion in the respective bifurcation (end portion 15) may thus be determined for example using the formula L_{Ductx}=L_{inserted}-L_{CC}.

Registration of the bile ducts may furthermore be adjusted using the constraint/information that the guidewire portion of length_{CC} is situated in the main duct, for example by using “EVAR guidance” methods.

Similarity measures may again now be determined using the following calculation approaches or combinations thereof: comparison of the angles/orientations of the line portions; Hausdorff metric; bringing the entire guidewire portion 14, 15 into register both with the combined portion of midlines 10 and 11 and with the combined portion of midlines 10 and 12 (truncated to the length of L_{inserted}).

Deformation methods for providing a visible tool in an invisible deformable vessel may again be used. Such elastic registration involving less deformation of the branches (bile ducts) is evaluated as a probable location of the end portion 15 of the guidewire 5.

The distances/metrics (similarity measures) between the complete guidewire portions 14, 15 inserted into the bile ducts and the combined midlines 10, 11 or 10, 12 may, for example, be compared. FIG. 3 indicates the distance measures as first distance 17 and second distance 18. In the present example, the distance 17 of the guidewire 14, 15 from the midline 10, 11 is greater than the second distance 18 from the combined midline 10, 11. The guidewire is therefore situated with a high level of probability in the second branch 9 with the second branch midline 12.

If the measure of probability is still too uncertain for the physician (thus for example only a 70% probability that the end portion of the guidewire is in the first branch), one of the following steps may be selected in order to increase the confidence with which the end portion 15 is assigned to the correct branch (bile duct): advancing the guidewire 5 a short distance; changing the X-ray angle and carrying out another posterior capture, since an additional perspective further restricts registration.

As soon as the probability measure has been sufficiently determined from a clinical standpoint (e.g., 90% probability that the end portion of the guidewire is in the common bile duct), the physician may advance the guidewire a greater distance and/or inject contrast agent.

There are patient anatomies in which the pancreatic duct and the common bile duct divide directly at the intestinal wall such that there is (virtually) no common channel. In this case, the length of the common channel (main duct) may be set to zero: L_{CC}=0.

The guidewire 5 may have a lateral, optically visible length scale/length marking. Using an optical camera in the duodenoscope, it is possible to “count along” during insertion of the guidewire into the bile ducts or the length of guidewire currently advanced into the bile ducts may be digitally evaluated in the video image. This is an alternative embodiment for determining L_{inserted}. Using this information, it is possible to determine whether the X-ray projection is substantially perpendicular to the axis of the guidewire.

The guidewire may have radiopaque markers that provide an additional option for comparing the true guidewire length with the corresponding length of the image of the guidewire in the X-ray image. The “viewing angle” onto the guidewire portion may thus be determined.

Instead of stating a probability for which bile duct the end portion of the guidewire is currently situated in, it is also possible from a specific probability threshold to state information as to which bile duct is more probable (without any indication of percentages).

Once the entire inserted length of the guidewire has been determined from a source of information other than the X-ray image, it is possible to estimate how steeply the guidewire is tilted relative to the X-ray projection axis by making a comparison with the length of the X-ray shadow of the guidewire portion in the X-ray projection image. This enables increased accuracy when carrying out the method according to embodiments and improved 2D/3D registration of the bile ducts on the basis of the guidewire's current position and shape.

FIG. 4 diagrammatically shows a process sequence of one embodiment of a method according to embodiments. The first two steps may or may not be performed. In a first step S1, an imaging modality creates a capture of a main duct and of a first and a second bifurcation. The capture is automatically processed in step S2. Image recognition is performed and/or center- or midlines of the respective ducts are determined.

A data set that represents the main duct and the bifurcations is provided in step S3. This data set is obtained from the first two steps S1 and S2.

A course of the medical instrument, for example a course of the midline of the medical instrument, is detected in a further step S4. According to step S5, the course of the medical instrument may now be brought into register with the course of the main duct from the data set from step S3.

After registration, similarity measures relating to the similarity of the course of the medical instrument with the courses of the two branches may be calculated according to step S6. Finally, according to step S7, an item of location information that includes a probability that the medical instrument is situated in one of the branches may be determined on the basis of the similarity measures.

It is to be understood that the elements and features recited in the appended claims may be combined in different ways to produce new claims that likewise fall within the scope of the present embodiments. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present embodiments have been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for locating a distal end of a medical instrument, the method comprising:
providing a data set that describes a course of a main duct, of a first branch from the main duct, and of a second branch from the main duct;
detecting a course of the medical instrument in the main duct and in one of the first branch or the second branch;
bringing the course of the main duct and of the first branch and the second branch into register with the course of the medical instrument;
calculating a first similarity measure of a portion of the course of the medical instrument in the first branch or the second branch with a first portion of the course of the first branch and a second similarity measure of the portion of the course of the medical instrument in the first branch or the second branch with a second portion of the course of the second branch; and
determining an item of location information about the portion of the course of the medical instrument from the first similarity measure and second similarity measure.

2. The method of claim 1, wherein the data set is created from at least one X-ray image, MRI image, a computed tomography (CT) image, and/or ultrasonogram.

3. The method of claim 1, wherein a length of the medical instrument is detected and the method is only carried out once the length of the medical instrument is greater than a length of the course of the main duct of the data set.

4. The method of claim 1, wherein the courses of the main duct and of the branches are respective 2D projections.

5. The method of claim 1, wherein the course of the main duct and the course of the first branch and second branch are saved in the data set as concatenated line portions.

6. The method of claim 1, wherein the first similarity measure and the second similarity measure are in each case based on an angle of the portion of the course of the medical instrument in the first branch or second branch in a predetermined coordinate system.

7. The method of claim 1, wherein the first similarity measure and the second similarity measure are in each case based on a Hausdorff metric between the portion of the course of the medical instrument in the first branch or second branch and the respective portion of the course of the respective branch.

8. The method of claim 1, wherein, on the basis of the similarity measures, the location information is calculated as a probability with which the medical instrument is situated in the first branch or second branch, and where the probability or a value based on the probability is provided to a user.

9. The method of claim 1, wherein a risk measure is determined as a function of a length of the medical instrument in the first branch or second branch and the similarity measures and is provided to a user.

10. The method of claim 1, wherein detection of the course of the medical instrument is carried out automatically by an imaging modality and a data processing facility.

11. The method of claim 1, wherein a length of the medical instrument is detected independently of the detection of the course of the medical instrument and used for calculating the similarity measures.

12. A system comprising:
a medical instrument;
a memory facility configured to provide a data set that describes a course of a main duct, of a first branch from the main duct, and of a second branch from the main duct;
an imaging modality configured to detect a course of the medical instrument in the main duct and in one of the first branch or the second branch; and
a data processing facility configured for:
bringing the course of the main duct and of the first branch and second branch into register with the course of the medical instrument;
calculating a first similarity measure of a portion of the course of the medical instrument in the first branch or the second branch with a first portion of the course of the first branch and a second similarity measure of the portion of the course of the medical instrument in the first branch or the second branch with a second portion of the course of the second branch; and
determining an item of location information about the portion of the course of the medical instrument from the first similarity measure and the second similarity measure.

13. The system of claim 12, wherein the medical instrument is part of a catheter, a sphincterotome, a duodenoscope, or a guidewire.

14. The system of claim 12, wherein the imaging modality comprises an X-ray apparatus, an MRI apparatus, a CT apparatus, or an ultrasonography apparatus.

15. A non-transitory computer implemented storage medium that stores machine-readable instructions for locating a distal end of a medical instrument executable by at least one processor, the machine-readable instructions comprising:
providing a data set that describes a course of a main duct, of a first branch from the main duct, and of a second branch from the main duct;
detecting a course of the medical instrument in the main duct and in one of the first branch or the second branch;
bringing the course of the main duct and of the first branch and the second branch into register with the course of the medical instrument;
calculating a first similarity measure of a portion of the course of the medical instrument in the first branch or the second branch with a first portion of the course of the first branch and a second similarity measure of the portion of the course of the medical instrument in the first branch or the second branch with a second portion of the course of the second branch; and
determining an item of location information about the portion of the course of the medical instrument from the first similarity measure and second similarity measure.
